(19) United States
(12) Patent Application Publication
(10) Pub. No.: US 2005/0203450 A1
(43) Pub. Date: Sep. 15, 2005

(54) Heated splint system

(76) Inventor: Ronald D. Shippert, Littleton, CO (US)

Correspondence Address: SHERIDAN ROSS PC, 1560 BROADWAY, SUITE 1200, DENVER, CO, 80202, US

(21) Appl. No.: 10/798,805

(22) Filed: Mar. 10, 2004

(51) Int. Cl. A61F 5/00

(52) U.S. Cl. 602/7; 602/14

(57) ABSTRACT

A medical kit, comprising a medical device, a removal member for positioning about the medical device, a bag for housing liquid, and a heating unit for heating the medical device. The removal member is positioned about the medical device and placed in the bag, and the bag is placed in the heating unit. The heating unit heats the liquid and medical device. The medical device, in an embodiment, is a nose splint having a number of holes and an adhesive on at least one side of the nose splint. The kit may also include a wrap member located about at least portions of the medical device and with the removal member located outwardly of the wrap member. The wrap member, in an embodiment, is attached to the adhesive and the removal member is not in contact with the adhesive.

### FIELD OF THE INVENTION

This invention is directed to a medical system and in particular to a medical system for heating a medical device which may include a splint.

### BACKGROUND OF THE INVENTION

Splinting is medically required in many situations. For example, splinting is necessary after certain fractures and medical procedures, including nasal bone fractures and rhinoplasty operations. The splint material may be one of any useful materials, the most common being aluminum models, plaster, and thermoplastic polymers such as polypropylene.

In cases where the nose is splinted, many surgeons prefer to splint with a thermoplastic polymer such as a heat-sensitive polypropylene mesh. Generally, such a heat-sensitive polypropylene mesh comes as a relatively large mesh sheet. Prior to splinting, a splint is cut from the mesh sheet. After the splint is cut, it is heated in order to make the splint malleable so a surgeon can form the splint properly. After heating and forming, the splint is attached to the area to be splinted. The splint may be attached by adhesion of the splint to the patient's skin, either by the natural adhesion of the heated polymer, or with an added adhesive.

While present day thermoplastic polypropylene splints are favorable to many other splinting materials, there are multiple problems associated with the use of thermoplastic polypropylene splints as currently used. For example, as mentioned above, the splint is generally cut from a large piece of mesh having pre-punched holes which provide ventilation to the underlying skin. This requires a surgeon, or an assistant, to spend time cutting the splint from the sheet and often results in wasted material. Furthermore, once the splint is cut from the sheet, the pre-punched holes result in a splint which has ragged edges. Such ragged edges can result in damage to the underlying skin in the areas of the ragged edges. Accordingly, it would be beneficial to have a polypropylene splint which reduces or eliminates skin damage associated with ragged edges of the splint.

Another problem with present day polymer splints is poor adhesion. Poor adhesion is often the result of oil build-up on the underlying skin caused by inadequate ventilation of the underlying skin. As mentioned above, present day splints are generally cut from a mesh sheet having pre-punched holes. The pre-punched holes usually account for about 20% of the surface area of the sheet. When attached to a person's nose, 20% air exposure of the underlying skin is many times not enough to provide adequate ventilation of the underlying skin, leading to oily skin which can reduce the adhesion of the splint, resulting in the splint coming off of the nose. Some surgeons attempt to use an adhesive on the underside of the splint. However, when added, such an adhesive can block some or all of the pre-punched holes, allowing even less air to get to the skin. Thus, the addition of adhesive to the splint can often result in a splint which, instead of staying on longer, actually reduces the time the splint will stay on the nose. Accordingly, it would be beneficial to have a splint which remains adhered to a patient's skin for a longer time than provided by present day splints.

In addition to problems related to damaging skin and coming off of skin relatively quickly, present day splints can also be difficult to properly heat and apply in an operating room. It is common to have a heat source for the splint which is in a remote location from the operating room. In these cases, typically an assistant is required to heat the splint in another area using a microwave or stove. Also, the splint is typically heated in a container holding water and the splint. This container commonly presents a set of problems as well. It is common for the splint, once heated, to stick to the container, thus requiring time and effort to remove the splint from the container. Furthermore, when heated, the splint typically becomes transparent making it very difficult to identify the splint in the water. Once heated, the splint must be located in the container of water and taken to the operating room where is must be formed into the proper shape before it cools too much to be effective. All of these factors result in splints commonly having to be re-heated, which in turn required additional time in the operating room. It is common for operating room costs to be more than several thousand dollars per hour, thus any additional time required as a result of the logistics of getting a properly heated splint to the surgeon can be very expensive. Accordingly, it would be beneficial to have a splint and a heating system which can be reliably identified and handled while heated, and which may be readily accessible in its heated state at the time required by the surgeon.

### SUMMARY OF THE INVENTION

The present invention addresses the aforementioned problems and meets the aforementioned needs. The present invention provides a method and apparatus for heating a medical device.

One aspect of the present invention provides a method for providing a heated medical device. The method includes providing a kit that includes the medical device, a bag for housing liquid and a removal member. The medical device, while in the hag, is heated, and the medical device is removed from the bag using the removal member. When providing the kit, the kit may be prepared for heating, including placing the liquid in the bag. Providing the kit may also include positioning the medical device within the removal member. In one embodiment, the kit includes a wrap member and providing the kit includes positioning the medical device within the wrap member and disposing the removal member outwardly of the wrap member and the medical device. Providing the kit, in another embodiment, includes positioning at least portions of the removal member outwardly of the bag. Heating the medical device may include using a heating unit and having at least portions of the removal member extending outwardly of the heating unit. In an embodiment, the heating unit includes at least one of a heating plate and a heating element and in which the bag contacts at least one of the heating plate and heating element during heating. While heating, the heating unit, in an embodiment, maintains a desired temperature using electrical power until removing the medical device. In another embodiment, the kit includes a wrap member and the medical device includes a nose splint having adhesive, and providing the kit includes wrapping the wrap member about the nose splint, including the adhesive thereof, wherein the adhesive is attached to the wrap member and the removal member is disposed about the wrap member and not in contact with the adhesive. In yet another embodiment, providing the kit includes forming the nose splint with a number of holes using a laser, and providing a substantially smooth outer surface of the nose splint using the laser.

Another aspect of the present invention provides a medical kit, comprising a medical device, a removal member for positioning about the medical device, a bag for housing liquid, and a heating unit for heating the medical device while the removal member is positioned about the medical device and at least portions of the removal member are located outwardly of the bag. The medical device, in one embodiment, is a nose splint having a number of holes and an adhesive on at least one side of the nose splint. The kit may also include a wrap member located about at least portions of the medical device and with the removal member located outwardly of the wrap member. The wrap member, in an embodiment, is attached to the adhesive and the removal member is not in contact with the adhesive. In an embodiment, the removal member is substantially rigid and at least portions thereof extend outwardly of the heating unit and the bag when the bag, containing the medical device and the removal member, is heated using the heating unit. The heating unit, in an embodiment, includes an opening that is substantially longer than it is wider in which the bag containing the medical device and removal member is inserted. In an embodiment, the heating unit includes at least one of a heating plate and a heating element and in which at least portions of the bag are in contact with at least one of the heating plate and heating element.

The above described features and advantages of the invention will become more apparent from a review of the following description, taken in conjunction with the accompanying drawings.

### BRIEF DESCRIPTION OF THE DRAWINGS

FIG. 1 is an illustration of a thermoplastic polymer splint and sheet placed in a bag according to an embodiment of the present invention;

FIG. 2 is an illustration of a splint placed in a sheet according to an embodiment of the present invention;

FIG. 3 is an illustration of a splint with an adhesive layer between at least portions of the splint and a wrap member in one embodiment of the present invention;

FIG. 4 is an illustration, partially in cross-section, of a heating unit with a bag, removal member, and splint, of an embodiment of the present invention;

FIG. 5 is a cross-sectional illustration of the heating unit of FIG. 4;

FIG. 6 is a cross-sectional illustration of a heating unit of one embodiment of the present invention;

FIG. 7 is a cross-sectional illustration of a heating unit of another embodiment of the present invention;

FIG. 8 is a cross-sectional illustration of a heating unit of still another embodiment of the present invention; and

FIG. 9 is a cross-sectional illustration of a heating unit of yet another embodiment of the present invention.

### DETAILED DESCRIPTION

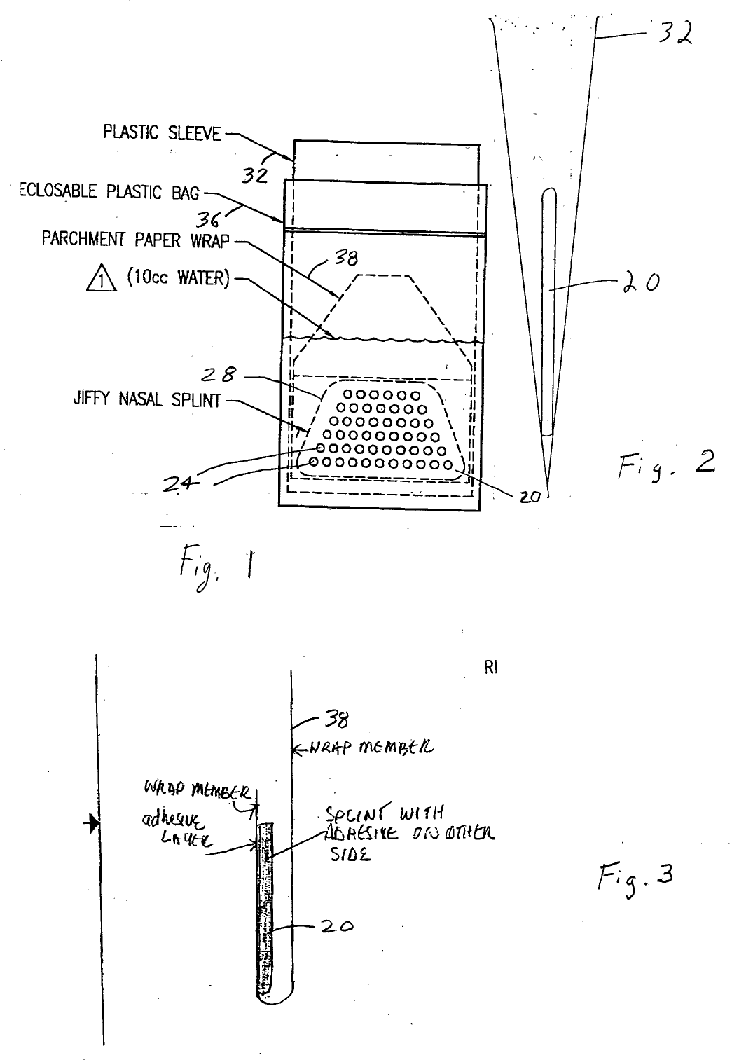

Referring to FIG. 1, a splint of one embodiment, and included with part of a system for heating it, is illustrated. The splint 20 is formed of a thermoplastic polymer, and in one embodiment is formed of polypropylene, although the present invention is not limited to polypropylene and may be made of any suitable heat sensitive material. The splint 20 includes a number of holes 24 arranged in a pattern such that none of the holes 24 intersect the outer edge of the splint 20. This arrangement of the holes 24 results in a perimeter 28 without holes resulting in a smooth edge of the splint 20. In this fashion, the splint 20 of the embodiment of FIG. 1 reduces damage to underlying skin which may be caused by splints having ragged edges. In one embodiment, the perimeter 28 is ⅛ inch around the entire periphery of the splint 20, although the perimeter 28 may be larger, or smaller, than ⅛ inch. Furthermore, the perimeter 28 may have differing dimensions based on the position on the splint 20, such as the top edge having a larger perimeter relative to the side and bottom edges. The splint 20, as illustrated in FIG. 1 has a trapezoid shape, however, the splint may have another desired shape as required for the body port being splinted, such as square, round, clover, rectangle, or any other shape as needed.

The splint 20, in one embodiment, is about 1/16 inch thick. However, the splint 20 may have differing thicknesses based on how much support is required for a particular procedure. For example, if a surgeon expects that there will be significant swelling in the area to be splinted, a thicker splint 20 may be utilized. For example, alternative thicknesses may be available such as 3/32 inch, ⅛ inch, and 5/32 inch thicknesses. The total area of the splint 20, in one embodiment, is about two square inches which is the approximate size required for many nasal splints. However, the area of the splint may be greater or smaller depending upon the application.

The holes 24 allow for aeration of the underlying skin. This aeration allows the underlying skin to have less oil build up than would be present if no holes were present. In one embodiment, the holes 24 occupy approximately 30% to 40% of the total surface area of the splint 20. In this manner, the skin underlying the splint 20 is provided with sufficient aeration to allow the splint 20 to stay on for a desired time, which is 5 to 10 days in most instances. The splint 20, being formed of thermoplastic polymer, has natural tackiness which allows the splint 20 to adhere to the underlying skin. The increased aeration of the splint 20 provided by the relative large area of holes 24 allows the underlying skin enough aeration to reduce the build-up of oil which often results in traditional splints not adhering to the skin for a long enough period of time. As mentioned above, many traditional splints formed from polypropylene mesh have only about 20% of the surface area of the splint with holes. This relatively small amount of aeration results in increased oil build-up and the splint coming off of the underlying skin before desired. In one embodiment, the holes 24 are formed in the splint 20 using a laser in a manner which gives adequate ventilation of the underlying skin while also providing an adequate amount of polymer in contact with the underlying skin to provide adequate adhesion.

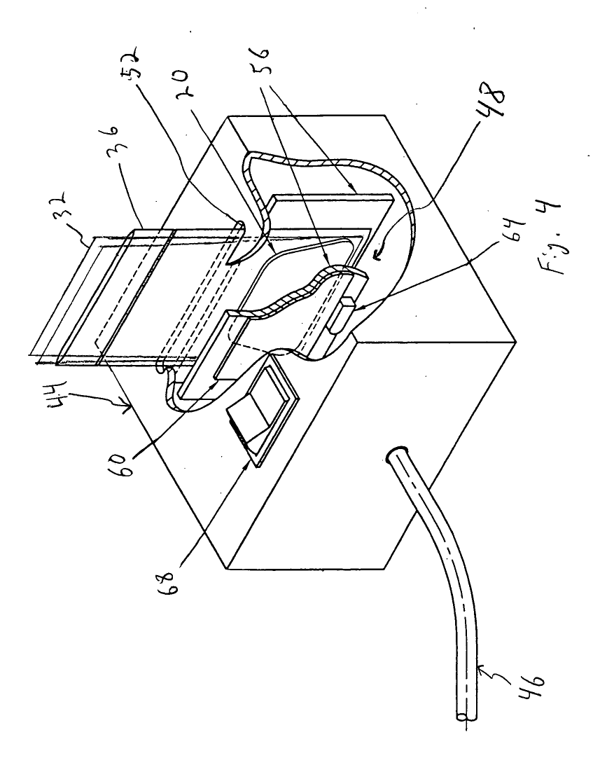

With reference to FIGS. 2- and 3, as well as FIG. 1, the system for preparing the splint 20 for application to a patient is described. As mentioned above, when preparing to apply the splint 20 to a patient, the splint 20 is heated such that the polymer may be formed or softened into the proper shape to provide adequate support for the body part being splinted, such as the nose. In order to assist in the identification and handling of a heated splint, in one embodiment, a removal member is provided which contains the splint 20. FIG. 2 illustrates an embodiment of the removal member. In this embodiment, the splint 20 is placed into a sheet or removal member 32. The sheet 32, in this embodiment, is a polypropylene sheet which is heat resistant to temperatures required to properly heat the splint 20. This allows the splint 20 to be heated within the sheet 32, and removed from the sheet 32 with little or no adhesion of the heated splint 20 to the sheet 32, or deformation of the sheet 32. The sheet 32, in one embodiment, is malleable at a temperature greater than 180 degrees Fahrenheit. The sheet 32, is sized to have a width slightly smaller than a bag 36, which will be described in more detail below, and to have a height which is slightly taller than the bag 36. In one embodiment, the splint 20 is placed within a wrap member 38, such as parchment wrap illustrated in FIGS. 1 and 3, which is then placed into the removal member 32. Adhesive is disposed between at least portions of the splint 20 and the wrap member 38 to define an adhesive layer.

To further assist in the heating and handling of the splint 20, in one embodiment the splint 20, together with the wrap member 38, and sheet 32 are placed in a plastic bag 36, as illustrated in FIG. 1. The bag 36 is also heat resistant to temperatures required to properly heat the splint 20. In one embodiment, the bag is a polypropylene bag which becomes malleable at temperatures greater than 180 degrees Fahrenheit. A preset amount of water, or other suitable liquid, is placed into the bag 36 along with the splint 20 and sheet 32. This liquid, within the bag 36 may then be heated, resulting in the splint 20 also being heated to a point where the splint 20 is able to be formed into the proper shape, while the bag 36 and sheet 32 maintain form through heating to allow for handling of the splint 20 after heating. The bag 36 may be a reclosable bag, and in one embodiment has a zipper closure 40. Thus the bag 36 may be filled with the appropriate amount of water and closed.

When heating of a splint 20 is required, the bag 36 may be opened and the liquid, such as water, the splint 20 and the sheet 32 may be placed inside the bag 36 and heated. In the embodiment, of FIG. 1, the sheet 32 extends beyond the top of the bag 36. This arrangement allows the sheet 32 and splint 20 to be removed from the bag without having to physically reach into the bag 36, which contains the hot liquid. However, in other embodiments the sheet 32 and splint 20 may be placed entirely within the bag 36, and the bag 36 may be sealed closed. In this manner, a prepackaged bag may be provided containing the splint 20, sheet 32, and water. This prepackaged bag may then be heated and the splint removed.

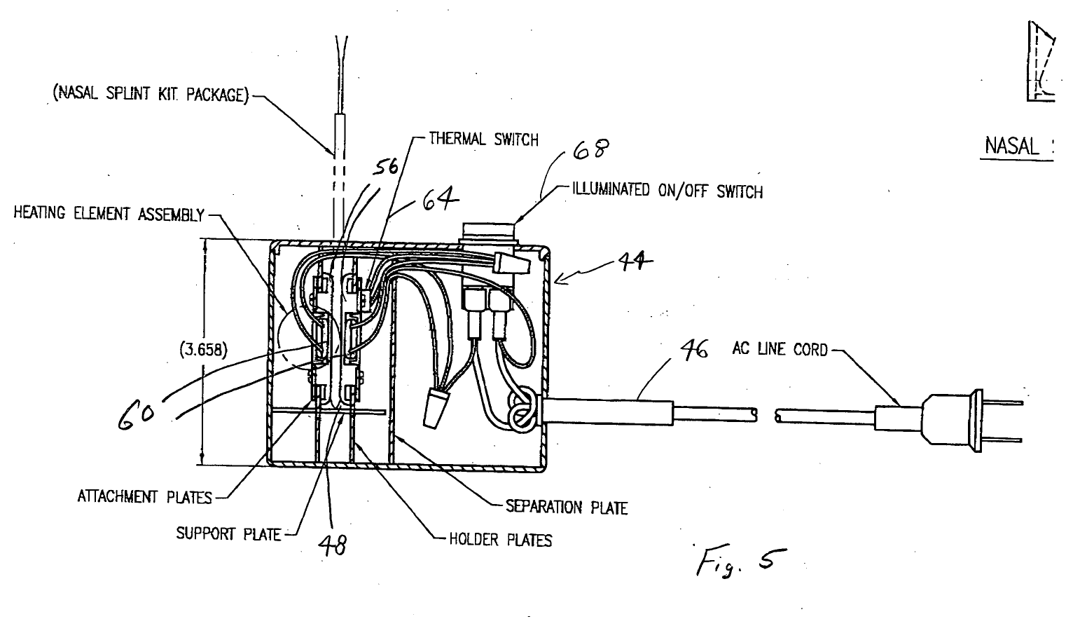

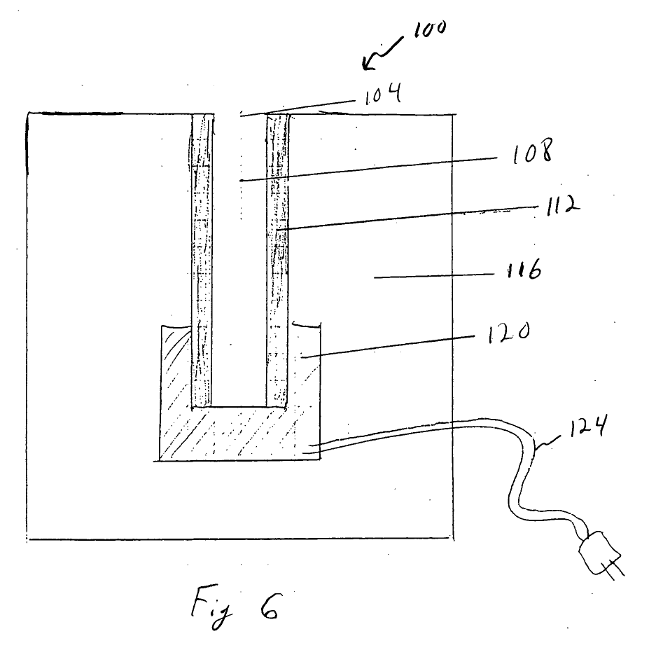

Several alternatives exist for heating the splint 20, sheet 32, bag 36, water and, optionally, the wrap member 38. In one embodiment, illustrated in FIGS. 4 and 5, the bag 36, sheet 32, splint 20, along with water added to the bag 36, are placed in a heating unit 44. The heating unit 44 is electric and connected to a standard electrical outlet through power cord 46. The heating unit 44 includes a heating chamber 48. The heating unit 44 has an opening 52 which provides access to the heating chamber 48. The opening in the embodiment of FIGS. 4 and 5 is designed such that the bag 36, including the splint 20 and sheet 32 is able to be fully placed into the heating chamber 48 with the top portion of the bag 36 positioned outside of the heating chamber 48. In this manner, the liquid within the bag 36 may be heated, thus heating the splint 20, and the splint 20 may be removed from the bag 36 by removing the sheet 32 from the bag 36. The bag 36, and heated liquid, thus remain within the heating chamber 48. Alternatively, the bag 36 is removed from the heating unit 44 with the sheet 32.

The heating unit 44, as illustrated in FIGS. 4 and 5, includes heating plates 56 having heating elements 60 attached to the heating plates 56. The heating elements 60 heat the heating plates 56, which provide heat to the heating chamber 48. The heating unit 44 may also include a thermal switch 64 which regulates the temperature of the heating chamber 48 at a predetermined temperature or within a predetermined temperature range. In one embodiment, the temperature range of the heating unit 44 is between about 160 and 180 degrees Fahrenheit. Thus, the heating unit 44 may be turned on at the switch 68, and left on until the splint 20 is required. The thermal switch 64 regulates the temperature of the heating chamber 48, keeping the heating chamber 48 hot enough to provide the adequate temperature to allow the splint 20 to be rendered soft, and preventing the heating chamber from becoming too hot and potentially causing heat deformation of the bag 36 or sheet 32. By keeping the heating chamber 48 within a preset temperature range for a period of time, the splint 20 may be heated in advance of the time it is required, and removed from the heating unit 44 at the time it is required, thus increasing efficiency and potentially reducing the total amount of operating room time required. In one embodiment, the heating unit 44 also includes a timer, which automatically turns off power to the heating unit 44 after a preset time period. Furthermore, the heating unit 44 may include an indicator which notifies personnel that the heating unit 44 has achieved the desired temperature, and thus that the splint 20 is ready to be formed into shape and applied to the patient. In one embodiment, the indicator is an audible bell which rings when the preset temperature is reached. The preset temperature, in an embodiment, is 180 degrees Fahrenheit, though this temperature can be adjusted based on the temperature the splint is to be heated to in order to properly form the splint. Other embodiments include a visual light, a voice alarm, or any other combination of visual and/or audible indications.

As discussed above, it is common for a thermoplastic polymer splint 20 to require additional adhesive in order to remain attached to underlying skin for relatively long periods of time. Such time periods correspond to the amount of time that additional support of a splint is required for the underlying body part, and may exceed the amount of time the splint 20 will typically remain adhered to skin due to the natural tackiness of the splint polymer material. In one embodiment, the liquid which is in the bag 36 contains a medical grade adhesive. This adhesive impregnates the splint 20 during heating of the splint 20, thus increasing the adhesion of the splint 20 to the underlying skin, and also does not interfere with the exchange of air to the skin. Alternatively, a separate adhesive film may be attached to one side of the splint 20. In one embodiment, this separate adhesive film is attached to the splint material prior to the creation of the holes 24 in the splint 20. In this manner, the splint 20 and adhesive film will have the same holes created, and the adhesive will not interfere with the exchange of air to the skin. The splint 20, in this embodiment, may be wrapped in the waterproof wrap member 38 to help prevent water from dissolving any of the adhesive film. Following the heating of the splint 20, the splint 20 and sheet 32 may be removed from the bag 36, the splint 20 and wrap member removed from the sheet 32, the splint 20 may then be removed from the wrap member, formed into the proper shape, and applied to the body part.

Referring now to FIGS. 6-9, some heat sources of other embodiments of the present invention are now described. The heating unit 100 of FIG. 6 includes a top opening 104 into which a splint and associated bag and sheet may be inserted. The heating chamber 108 has heat plates 112 which have integrated heating elements. The heating unit 100 has a dead space 116 which surrounds the heating chamber 108 and heat plates 112. A transformer and control unit 120 is interconnected with the heat plates 112 and heat chamber 108 to provide power to the heat plates 112 and temperature control to limit the maximum temperature achieved in the heat chamber 108. A power cord 124 is provided to connect the transformer and control unit 120 to a power source.

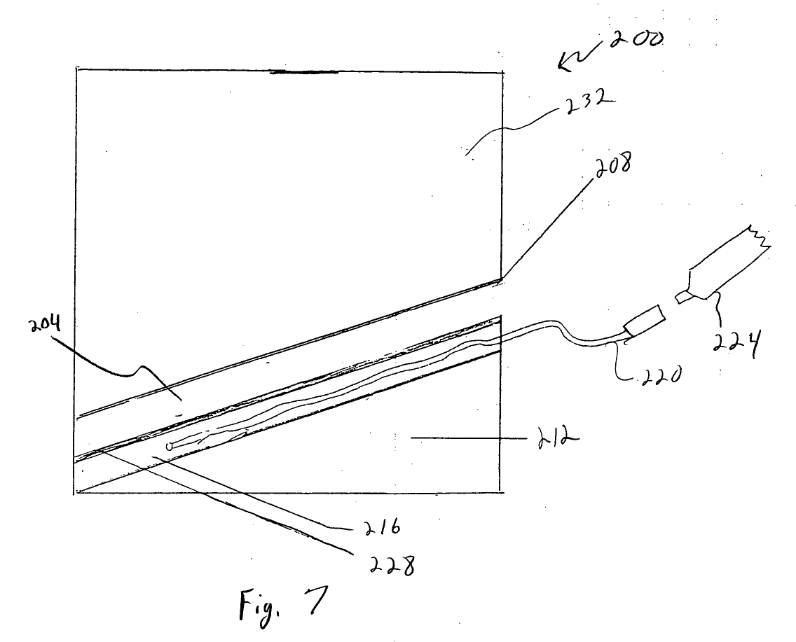

FIG. 7 illustrates a heating unit 130 of another embodiment of the present invention. In this embodiment, the heating unit 130 includes a top opening 134 which provides access to a heat chamber 138. The heat chamber 138 is heated using high intensity heating bulbs 142, with the radiation from the bulbs 142 heating the heat chamber 138. The heating unit 130 contains a dead space 146, and the bulbs 142 are connected to a power source through power cord 150. The heating unit 130 may also include a control unit which regulates the power to the bulbs 142, thus controlling the maximum temperature achieved in the heat chamber 138.

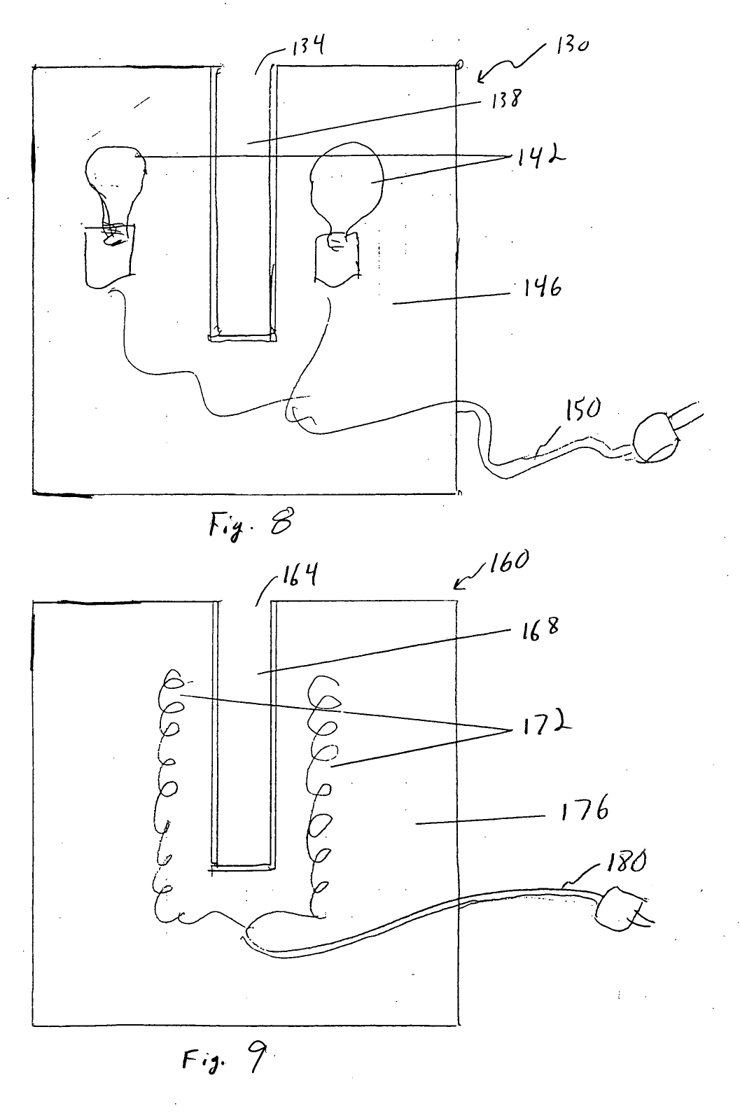

FIG. 8 illustrates a heating unit 160 of another embodiment of the present invention. In this embodiment, the heating unit 160 includes a top opening 164 which provides access to a heat chamber 168. The heat chamber 168 is heated using electrical coils 172, with the radiation from the coils 172 heating the heat chamber 168. The heating unit 160 contains a dead space 176, and the coils 172 are connected to a power source through power cord 180. The heating unit 160 may also include a control unit which regulates the power to the coils 172, thus controlling the maximum temperature achieved in the heat chamber 168.

It should be noted that the heating units described with respect to FIGS. 4-7 may alternatively receive power from other sources. For example, the heating units may contain a battery which supplies power to the heating unit. Such a battery may be replaced periodically, or may be rechargeable. The heating units may also receive power from any other available and suitable power source.

Referring now to FIG. 9, a disposable heating unit 200 of one embodiment of the present invention is now described. In this embodiment, the heating unit 200 has a heating chamber 204 having a side opening 208 for accessing the heat chamber 204 and into which a bag and splint may be inserted. The heat chamber 204 is heated by a chemical heat source. The chemical heat source of this embodiment is a disposable exothermic chemical heat source similar to heat sources utilized in military type MREs. Heat is produced by the reaction of magnesium and iron in the presence of electrolytes. The heating unit 200 includes a chemical chamber 212 with a batting or pad which contains magnesium and iron with electrolytes. In the chemical reaction, the magnesium functions as an anode and the iron functions as a cathode. Water is introduced to a water chamber 216, which activates the electrolyte resulting in corrosion of the metal particles in the chemical chamber 212 occurs. Water is introduced to the water chamber 216 through a tube 220 interconnected with the water chamber 216. A syringe 224 is used to add water in one embodiment. The byproducts of the chemical reaction are heat, magnesium hydroxide, and hydrogen gas. Between the heat chamber 204 and water chamber 216 is a perforated wall 228 which connects with the heat chamber 204 and allows hot gas and heat into the heat chamber 204. Above the heat chamber 204 is a batting 232 which can absorb moisture, gas, and residual heat from the reaction as they pass through the perforated wall 208. A heating unit 200 of this embodiment is thus portable and self-contained, allowing for use in the field away from any ready source of electrical power. In one embodiment, the heating unit 200, splint, sheet, and bag are packaged together, along with water ampules having water to place in the bag with the splint and to supply the water to activate the heating unit.

In one embodiment, the splint, and associated components are included in a kit. In this embodiment, the kit includes the polymer splint, the bag, the removal member, along with skin cleaner, skin protectant, skin adhesive, and absorbent tape. The kit may also include an alcohol sponge, a gauze type dressing, and a nose pack, such as a PVA foam pack. The kit may further include a disposable heat source, thus providing equipment to splint a nose in the field.

The foregoing discussion of the invention has been presented for purposes of illustration and description. Further, the description is not intended to limit the invention to the form disclosed herein. Consequently, variations and modifications commensurate with the above teachings, within the skill and knowledge of the relevant art, are within the scope of the present invention. The embodiments described hereinabove are further intended to explain the best modes presently known of practicing the inventions and to enable others skilled in the art to utilize the inventions in such, or in other embodiments, and with the various modifications required by their particular application or uses of the invention. It is intended that the appended claims be construed to include alternative embodiments to the extent permitted by the prior art.

1. A method for providing a heated medical device, comprising:
providing a kit that includes said medical device, a bag for housing liquid and a removal member;
heating said medical device while in said bag; and
removing said medical device from said bag using said removal member.

2. The method of claim 1 wherein said providing includes preparing said kit for heating including placing said liquid in said bag.

3. The method of claim 1 wherein said providing includes positioning said medical device within said removal member.

4. The method of claim 3 wherein said kit includes a wrap member and said providing includes positioning said medical device within said wrap member and disposing said removal member outwardly of said wrap member and said medical device.

5. The method of claim 1 wherein said providing includes positioning at least portions of said removal member outwardly of said bag.

6. The method of claim 1 wherein said heating includes using a heating unit and having at least portions of said removal member extending outwardly of said heating unit.

7. The method of claim 6 wherein said heating unit includes at least one of a heating plate and a heating element and in which said bag contacts said at least one of said heating plate and said heating element during said heating.

8. The method of claim 1 wherein said heating includes maintaining a desired temperature using electrical power until said removing.

9. The method of claim 1 wherein said kit includes a wrap member and said medical device includes a nose splint having adhesive and said providing includes wrapping said wrap member about said nose splint, including said adhesive thereof, wherein said attached adhesive is attached to said wrap member and said removal member is disposed about said wrap member and not in contact with said adhesive.

10. The method of claim 9 wherein said providing includes forming said nose splint with a number of holes using a laser and said forming includes providing a substantially smooth outer surface of said nose splint using said laser.

11. A medical kit, comprising:
a medical device;
a removal member for positioning about said medical device;
a bag for housing liquid; and
a heating unit for heating said medical device while said removal member is positioned about said medical device and at least portions of said removal member are located outwardly of said bag.

12. The kit of claim 11 wherein said medical device includes a nose splint having a number of holes and adhesive on at least one side of said nose splint.

13. The kit of claim 11 further including a wrap member located about at least portions of said medical device and with said removal member located outwardly of said wrap member.

14. The kit of claim 13 wherein said wrap member remains detached from said adhesive and said removal member is not in contact with said adhesive.

15. The kit of claim 11 wherein said removal member is substantially rigid and at least portions thereof extend outwardly of said heating unit and said bag when said bag, containing said medical device and said removal member, is heated using said heating unit.

16. The kit of claim 11 wherein said heating unit includes an opening that is substantially longer than it is wider in which said bag containing said medical device and said removal member is inserted.

17. The kit of claim 11 wherein said heating unit includes at least one of a heating plate and a heating element and in which at least portions of said bag are in contact with at least said one of said heating plate and said heating element.
